(19) United States
(12) Patent Application Publication
(10) Pub. No.: US 2008/0288074 A1
(43) Pub. Date: Nov. 20, 2008

(54) INTERNALLY REINFORCED ELASTOMERIC INTERVERTEBRAL DISC IMPLANTS

(76) Inventors: Michael J. O'Neil, West Barnstable, MA (US); Hassan Serhan, S. Easton, MA (US); Seung kyu Daniel Kwak, Grafton, MA (US); Steve Connolly, Sharon, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/748,755

(22) Filed: May 15, 2007

(51) Int. Cl. A61F 2/44; A61F 2/28

(52) U.S. Cl. 623/17.16; 623/17.15; 623/23.58

(57) ABSTRACT

Two techniques to minimize intervertebral disc height loss due to polymer creep following implantation of an elastomeric disc with the use of internal reinforcement are disclosed. The specific devices and methods relate to use of (i) singular or multiple internal reinforcement members to increase construct stiffness, reduce peripheral bulging and corresponding height loss; and (ii) internal elastomer fillers of various shapes which are compounded into the elastomer to share the load and constrain the elastomeric material from excessive deformations.

### BACKGROUND OF THE INVENTION

1. Field of the Invention

This invention is concerned with devices used to repair intervertebral discs and more particularly with intervertebral disc replacement devices that are less susceptible to permanent disc height loss.

2. Related Art

Many elastomeric disc replacement devices have been contemplated in the art for implantation in animal models and in humans. The art discloses from solid rubber devices articulating on the vertebral bodies, solid elastomeric devices integrally attached to device endplates or elastomeric devices articulating on device endplates. Several mechanical means to prevent excessive height loss are disclosed within the current art including positive stops, spring reinforcements, variable durometers materials, concentrically placed reinforcement fabrics (radial tires) and external containment methods like balloons, pistons, walls, for example. The concept of internal laminate layers for elastomers has been utilized in other non-medical industries to prevent creep and permanent deformations; one notable example is skyscraper isolation mounts.

In the repair of intervertebral discs, US20050171611 A1 discloses the use of dehydrated polymers(hydrogels) to regain disc height. More specifically this publication focuses on the use of dehydrated polymers(hydrogels) with internal polyethylene teraphthalate layers to constrain hydrogel growth to a vertical direction for regaining disc height. This publication also discloses a method of prosthesis production, which comprises: prefabricating soft and rigid layers; stacking at least two prefabricated soft and at least one prefabricated rigid layer in a parallel fashion into their final form; and, permitting the layers to firmly connect to one another by mutual interaction.

U.S. Pat. No. 4,911,718 discloses a disc spacer having a central biocompatible elastomeric core circumferentially wrapped by several layers of laminae. The laminate comprises strips of sheets of reinforcement fibers embedded in a biocompatible elastomer. However, there appears to be no disclosure of arranged reinforcement members in a generally horizontal orientation to restrain the elastomer as hereinafter described.

This invention differs from the closest prior art as it employs specific internal reinforcement layers or fillers to reduce material deformations of elastomeric polymers.

### BRIEF DESCRIPTION OF THE DRAWINGS

FIGS. 1A and 1B represent an elastomeric intervertebral disc replacement device.

FIGS. 1C and 1D represents an elastomeric intervertebral disc replacement device containing a reinforcement member but no endplates.

FIGS. 2A and 2B depict an elastomeric intervertebral disc replacement device containing reinforcement members.

FIGS. 3A and 3B represent an elastomeric intervertebral disc replacement device containing reinforcement members of a concave design.

FIGS. 4A and 4B depict an elastomeric intervertebral disc replacement device containing reinforcement members in the form of donut-shaped laminates.

FIGS. 5A and 5B depict an elastomeric intervertebral disc replacement device containing reinforcement members in the form of crescent-shaped laminates.

FIGS. 6A and 6B represent an elastomeric intervertebral disc replacement device containing a concave elastomer with a laminate reinforcement member contained therein.

FIGS. 7A and 7B depict an elastomeric intervertebral disc replacement device containing a concave elastomer with two reinforcement members extending to the periphery of the elastomer.

FIGS. 8A and 8B represent an elastomeric intervertebral disc replacement device containing reinforcement members designed to contact or nearly contact one another.

FIGS. 9A and 9B depict an elastomeric intervertebral disc replacement device containing reinforcement members designed to contact or nearly contact one another.

FIGS. 10A and 10B represent an elastomeric intervertebral disc replacement device containing reinforcement members designed to nest or nearly rest among one another.

FIGS. 11A and 11B represent an elastomeric intervertebral disc replacement device containing reinforcement members designed to nest or nearly rest among one another.

FIGS. 12A and 12B represent an elastomeric intervertebral disc replacement device containing reinforcement members of an interlocking platelet shape.

FIGS. 13A and 13B depict an elastomeric intervertebral disc replacement device containing reinforcement members of a disc or flake design.

FIGS. 14A-D depict various filler shapes capable of being used as reinforcement members.

### SUMMARY OF THE INVENTION

This invention differs from the closest prior art as it employs specific internal reinforcement layers or fillers to reduce material deformations of elastomeric polymers in an intervertebral disc replacement device.

Thus one aspect of the invention relates to an intervertebral disc replacement device comprising:
a) an elastomeric core comprising at least one reinforcement member set in a substantially horizontal orientation; and
b) optionally, a top endplate and a bottom endplate attached to the respective top and bottom ends of the elastomeric core.

Another aspect of the relates to an intervertebral disc replacement device comprising:
a) an elastomeric core comprising filler materials; and
b) optionally, a top endplate and a bottom endplate attached to the respective top and bottom ends of the elastomeric core.

Additional aspects of this invention relate to methods of manufacture of the intervertebral replacement discs comprising elastomeric cores with reinforcement members and/or fillers.

### DETAILED DESCRIPTION OF PREFERRED EMBODIMENTS OF THE INVENTION

Elastomeric discs are subject to both short term and long term deformations upon anatomical loading. The elastomers can be either theromset as is in the case of polyhexene (AcroFlex Bion rubber), silicones, polyol/issocyanate urethanes, for example; or can be a thermoplastic elastomers (TPE's) as in the case of thermoplastic urethanes (aromatic and aliphatic), as well as block co-polymers like polycarbonate urethanes (Bionate PCU), polycarbonate-silicone urethanes (Pursil), for example. Additionally the elastomer can be a solution cast material as in the case of the polyurethane elastomer sold under the trade name of Elasteon HF.

This invention contemplates internal reinforcement members to internally restrain the elastomer from radial deformations. This restraining force increases construct stiffness, reduces peripheral bulging and corresponding height loss. The internal reinforcement members can be placed at a generally horizontal orientation to restrain the elastomer. Reinforcement members can be comprised of varying materials, geometries and locations to enhance resistance to deformation to the desired directional forces. The internal reinforcement members may be produced from materials of various degrees of stiffness and range from relatively rigid metallics (CoCr, SS, for example) to more flexible metallics (Nitenol, for example) or elastomeric polymers (polyurethanes, silicones, for example, that recover following loading to minimize permanent deformation of the device). Specific material families for the reinforcement members include metallics, polymerics, and ceramics. The internal laminate layers can be of varying shapes including strips, donut-shapes, discs, squares, ovals, for example, and various fabricated sheets, woven or non-woven fabrics, porous/non-porous foams, for example. The layers can be produced from the following materials: metallics including SS, CoCr, CpTi, Ti6Al4V, SS, TiN, for example, as well as polymeric and ceramic materials including Ti, Ta, SS, CoCr, Calcium, Barium, PEEK, PU, PMMA, Clays, Talc, Mica, TiO2, for example. The reinforcement layers can extend to the periphery of the device or may be contained within the elastomer. The reinforcement layers can have protruding and receiving geometries that contact and even interlock to further reduce height deformation and loss. Some specific embodiments of the foregoing are illustrated in the following figures.

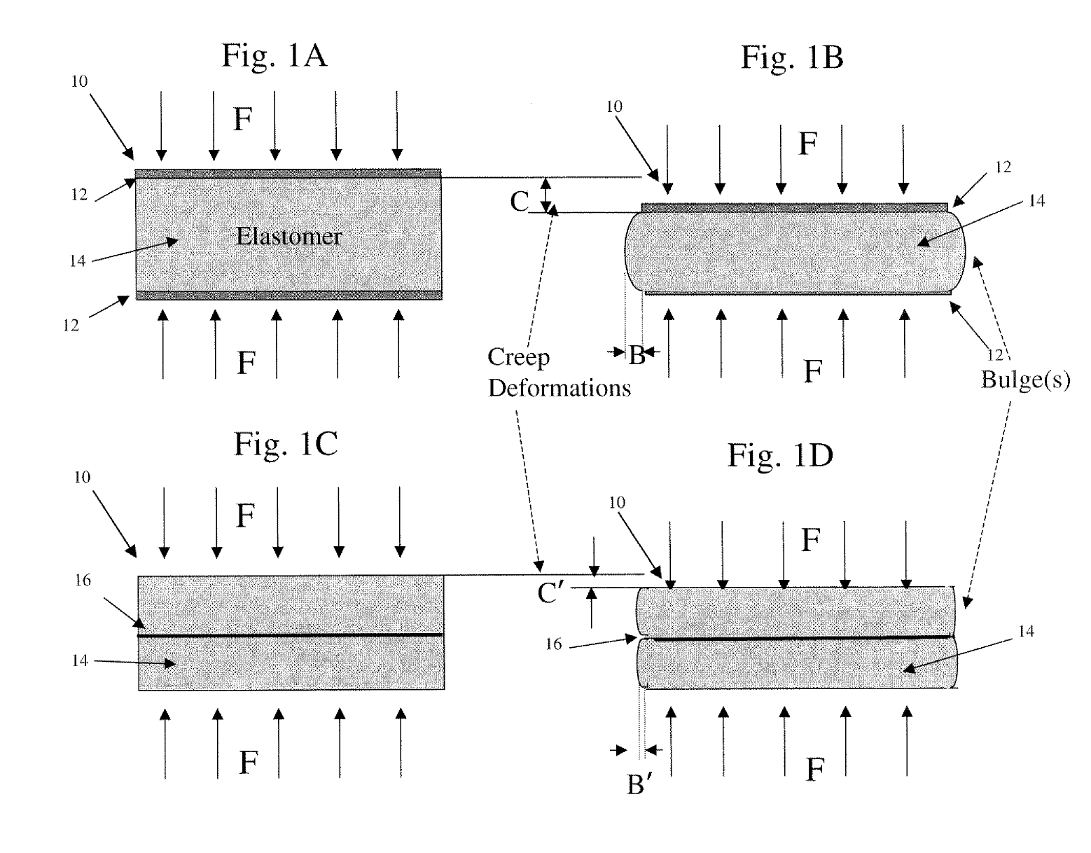

FIGS. 1A and 1B represent an intervertebral disc replacement device 10 comprising endplates 12 and elastomeric material 14. As force F is applied, elastomeric material 14 compresses and bulges as depicted in FIG. 1B. Also identified in FIG. 1B is the dimension C which represents the loss in disc height which results from elastomer 14 not able to return to its original orientation (“creep deformation”).

FIGS. 1C and 1D depict device 10 further comprising a reinforcement member 16 but with no endplates. The benefits of reinforcement member 16 can be seen by comparing FIG. 1D with FIG. 1B. Specifically, one observes that creep deformation C′ of FIG. 1D is less than creep deformation C of FIG. 1B. This result is beneficial as the reinforcement member 16 has the effect of lessening intervertebral disc height loss. A further benefit of the use of reinforcement member 16 is that the “bulging” of elastomeric material 14 is lessened as depicted in FIG. 1D as compared to FIG. 1B. In effect, the series of smaller bulges in FIG. 1D has less of a pronounced bulge as compared to the large bulges of FIG. 1B.

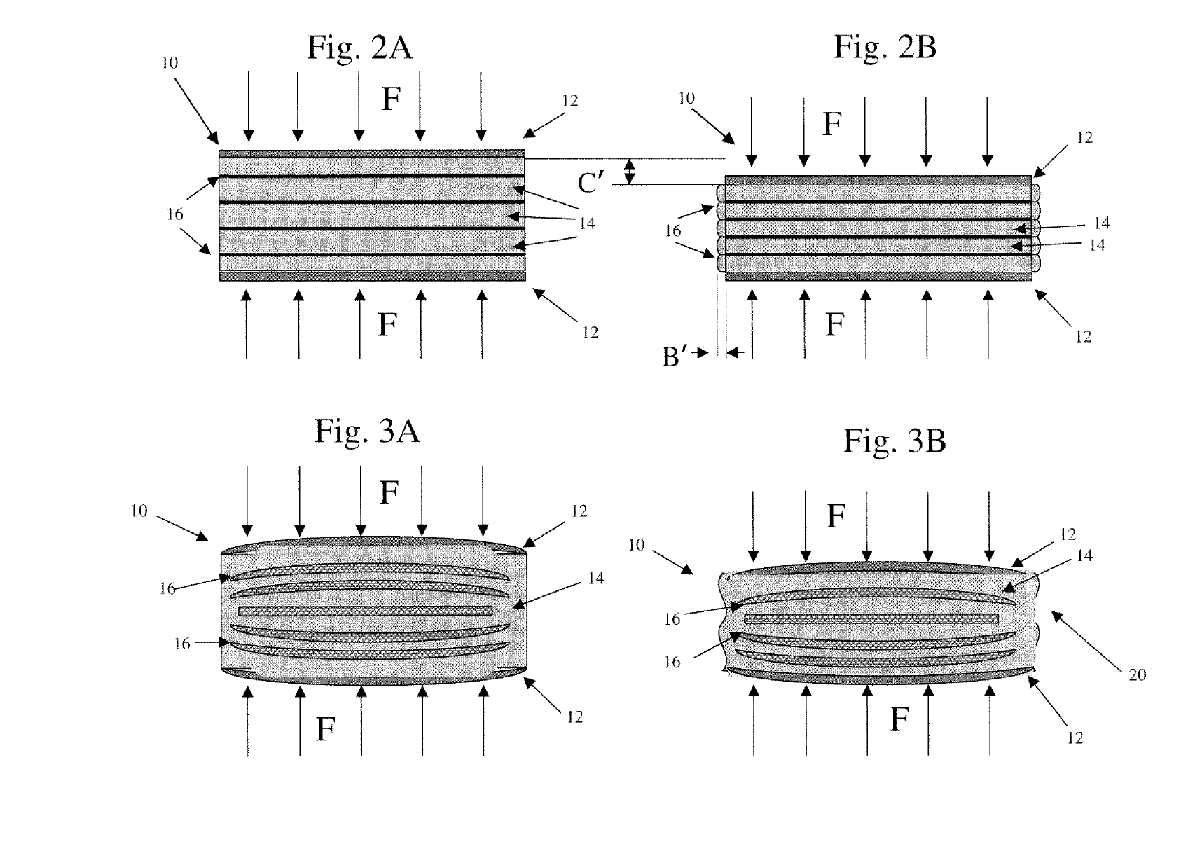

FIGS. 2A and 2B depict device 10 further comprising several reinforcement members 16 and endplates 12. The benefits of reinforcement members 16 can be seen by comparing FIG. 2B with FIG. 1B. Specifically, one observes that creep deformation C′ of FIG. 2B is less than creep deformation C of FIG. 1B. This result is beneficial as the reinforcement members 16 have the effect of lessening intervertebral disc height loss. A further benefit of the use of reinforcement members 16 is that the “bulging” of elastomeric material 14 from endplates 12 is lessened as depicted in FIGS. 2B as compared to FIG. 1B. In effect, the series of smaller bulges in FIG. 2B has less of a pronounced bulge as compared to the large bulges of FIG. 1B.

FIGS. 3A and 3B depict additional shapes for endplates 12 and reinforcement members 16. Specifically, endplates 12 are shown as “domed” which would allow device 10 better conform with the anatomy of the adjacent intervertebral body endplates. Furthermore, device 10 is also shown to contain reinforcement members 16 of varying shapes, including “dome” shaped members. Thus, the foregoing features represent alternate embodiments for endplates 12 and reinforcement members 16 as contemplated by the invention.

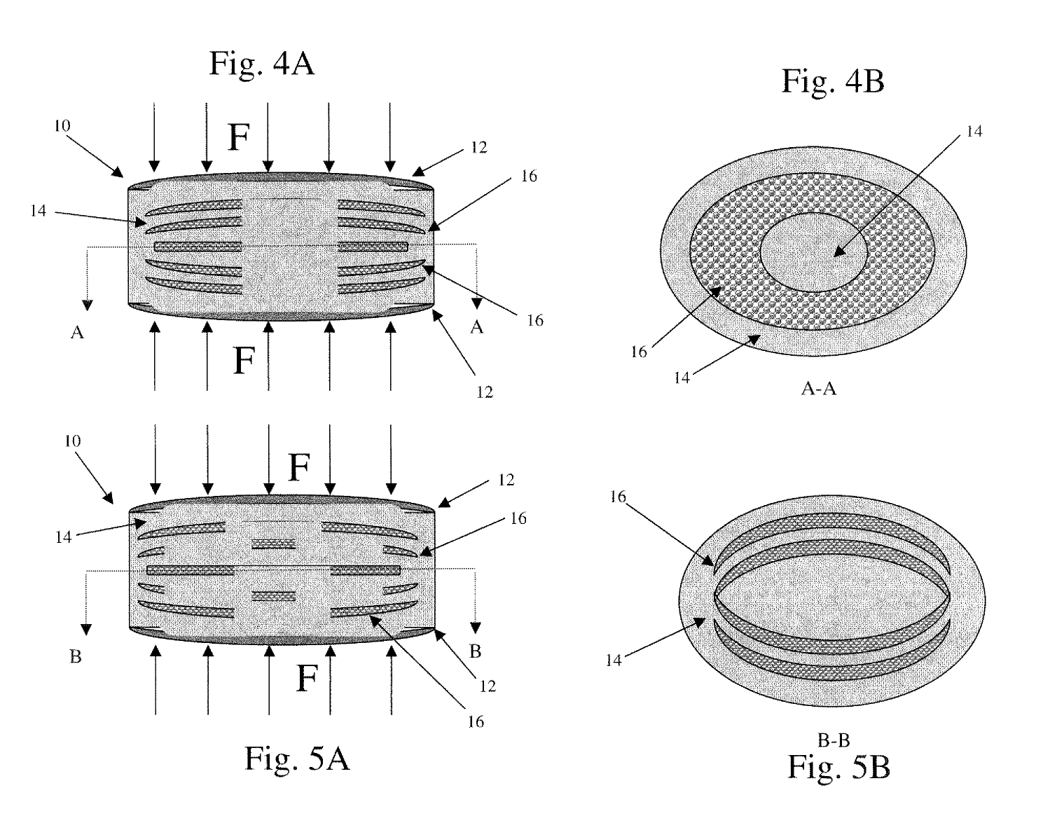

Further reinforcement member embodiments are depicted in FIGS. 4A and 4B and FIGS. 5A and 5B. In particular the reinforcement members 16 of FIGS. 4A and 4B are depicted as donut-shaped. The reinforcement members 16 of FIGS. 5A and 5B are shown as crescent-shaped.

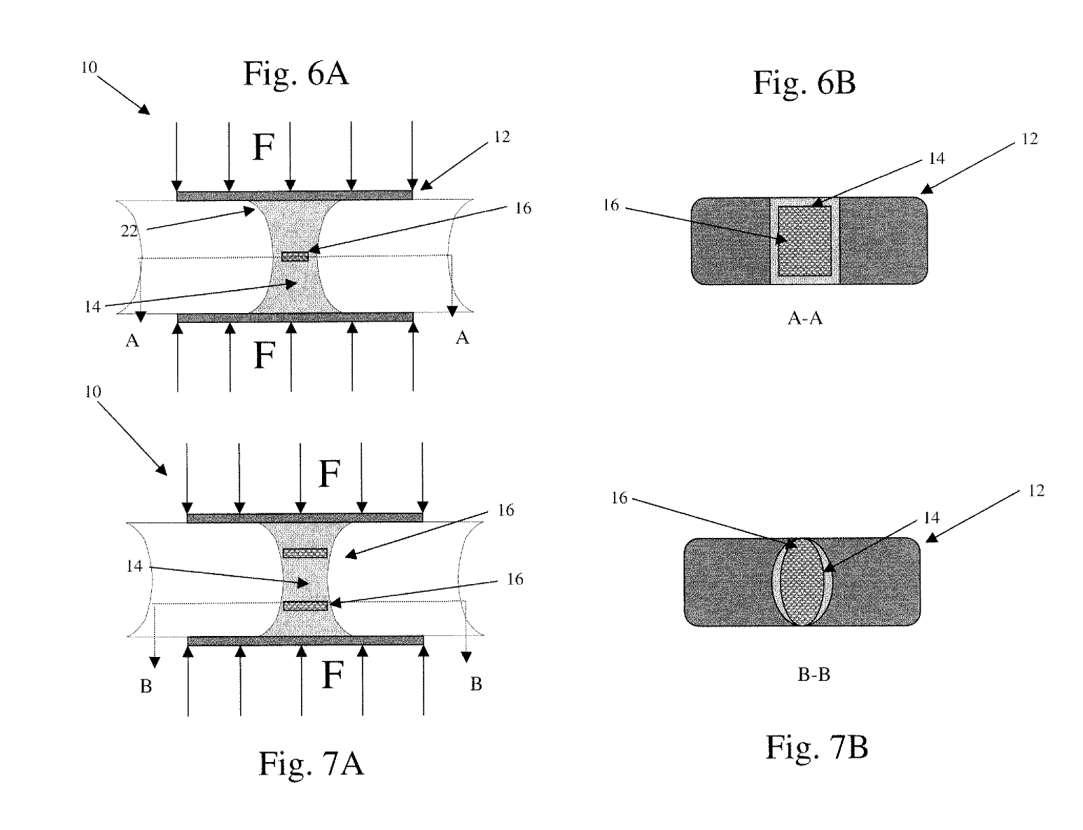

FIGS. 6A and 6B and FIGS. 7A and 7B depict alternate embodiments for device 10. In these figures, elastomeric material 14 has a concave shape 22 and includes reinforcement members 16 either as a laminate layer fully contained within elastomer material 14 (FIGS. 6A and 6B) or as laminate layers that extend to the peripheral edge of elastomeric material 14 (FIGS. 7A and 7B).

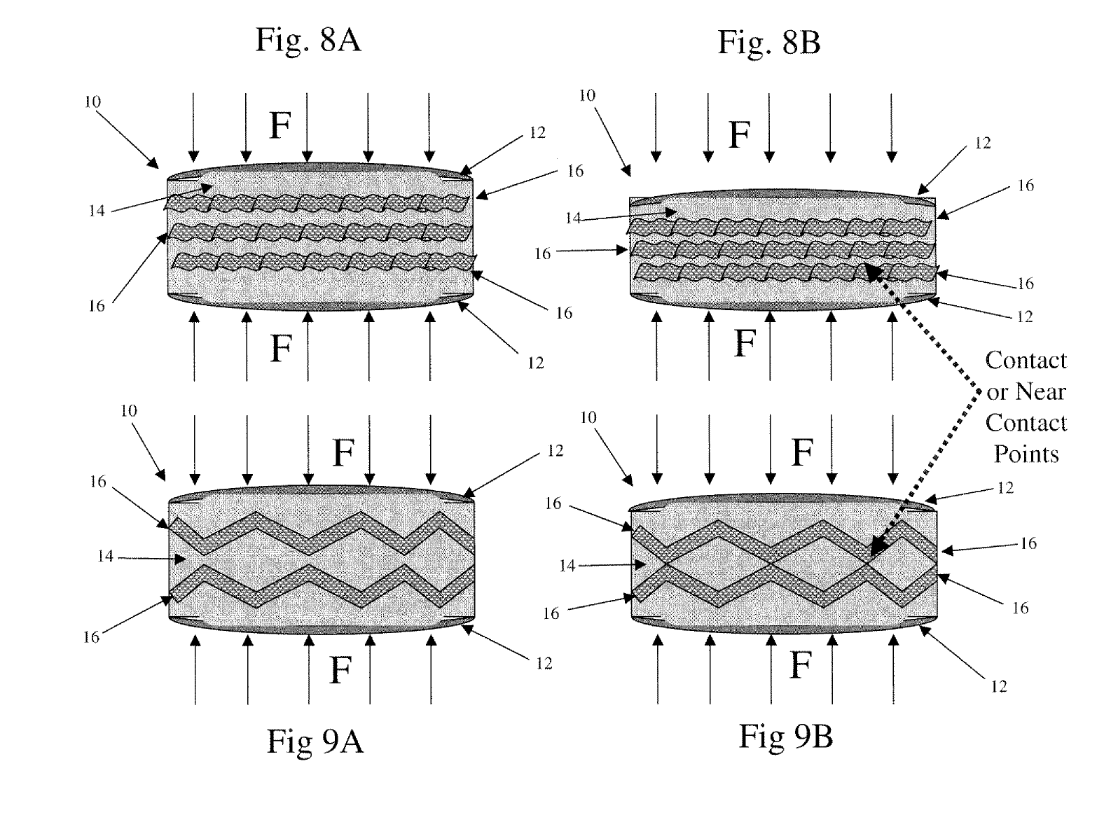

Yet further embodiments of device 10 are depicted in FIGS. 8A and 8B and FIGS. 9A and 9B. Generally, these figures depict embodiments where reinforcement member 16 are shown as laminates that are arranged to have a point(s) of contact or near point(s) of contact among and between one another. These configurations aid in alleviating disc height loss by approximating “stops” when reinforcement members 16 contact or nearly contact each other.

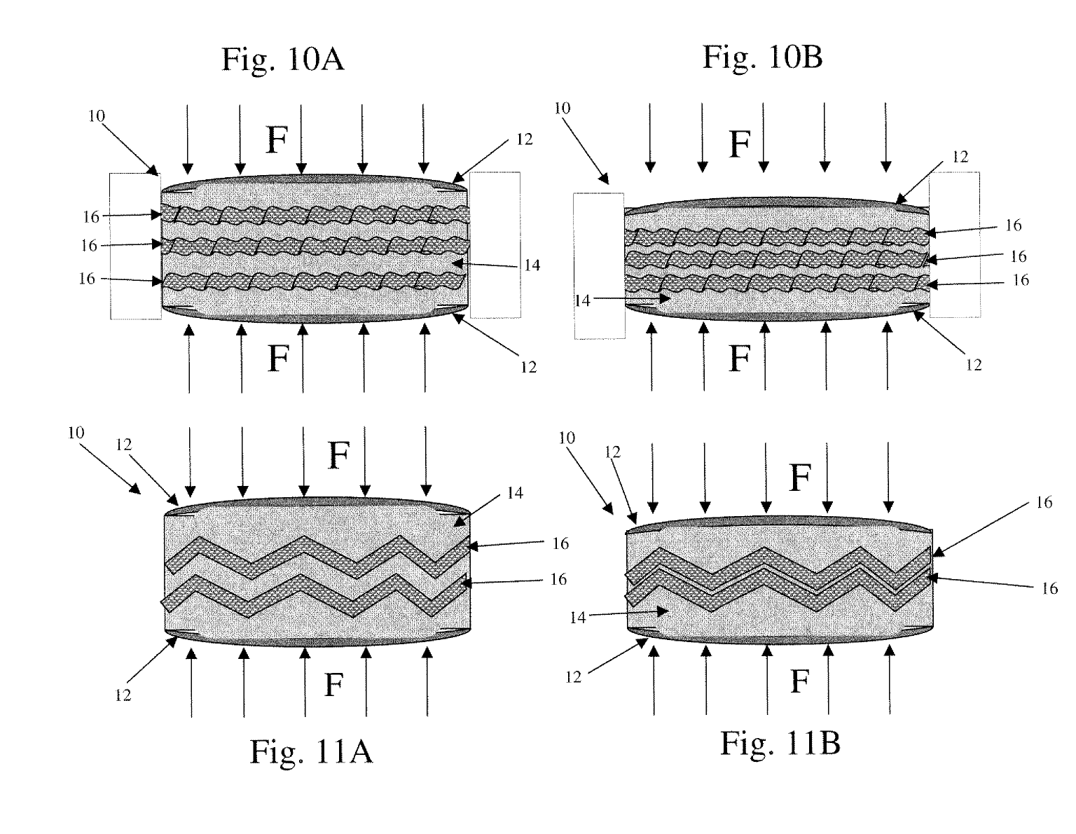

FIGS. 10A and 10B and FIGS. 11A and 11B represent additional embodiments of the invention wherein device 10 comprises nesting or near nesting reinforcement members 16. In general, FIGS. 10B and 11B show how reinforcement members 16 “nest” within one another and thus limit the potential amount of disc loss height and elastomeric material 14 deformation.

This invention further contemplates internal filler materials based on the internal restrain philosophy disclosed above, i.e., the principle of internal reinforcement with macro-laminate layers is applicable to the more micro-scale fillers. Increasing surface area on the fillers that are well bonded to the elastomer increases the constraint upon the elastomer matrix, minimizing bulging and permanent deformations. The fillers can be placed in random orientations, but preferably in a generally horizontal direction to minimize radial bulging and creep. Filler shapes include flakes, platelets, spheroids, spherlites, microspheres, micro-tubes, small disc, or other random external geometries. The fillers can be porous to allow for elastomer intrusion and increase surface for containment or can have interlocking features to providing filler based positive stop to deformations. The filler can be either organic or inorganic and fabricated from the following materials: metallics including SS, CoCr, CpTi, Ti6Al4V, SS, TiN, for example, as well as polymeric and ceramic materials including Ti, Ta, SS, CoCr, calcium, barium, PEEK, PU, PMMA, clays, talc, mica, TiO2, for example.

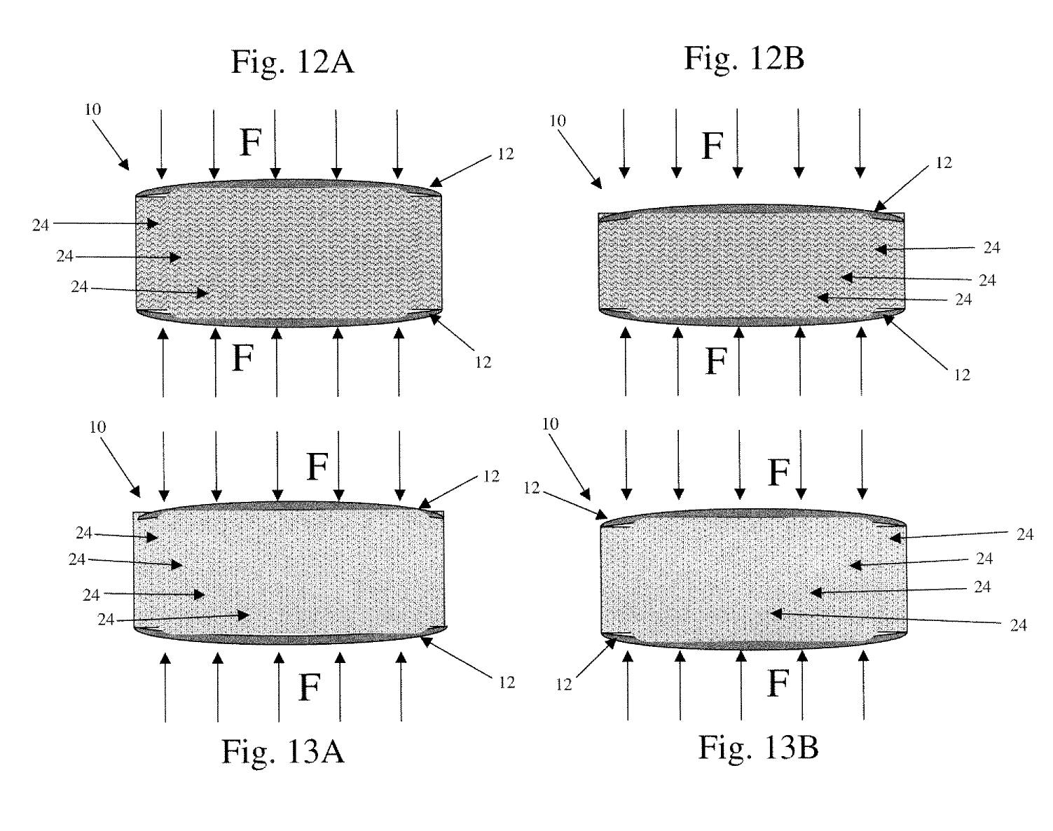

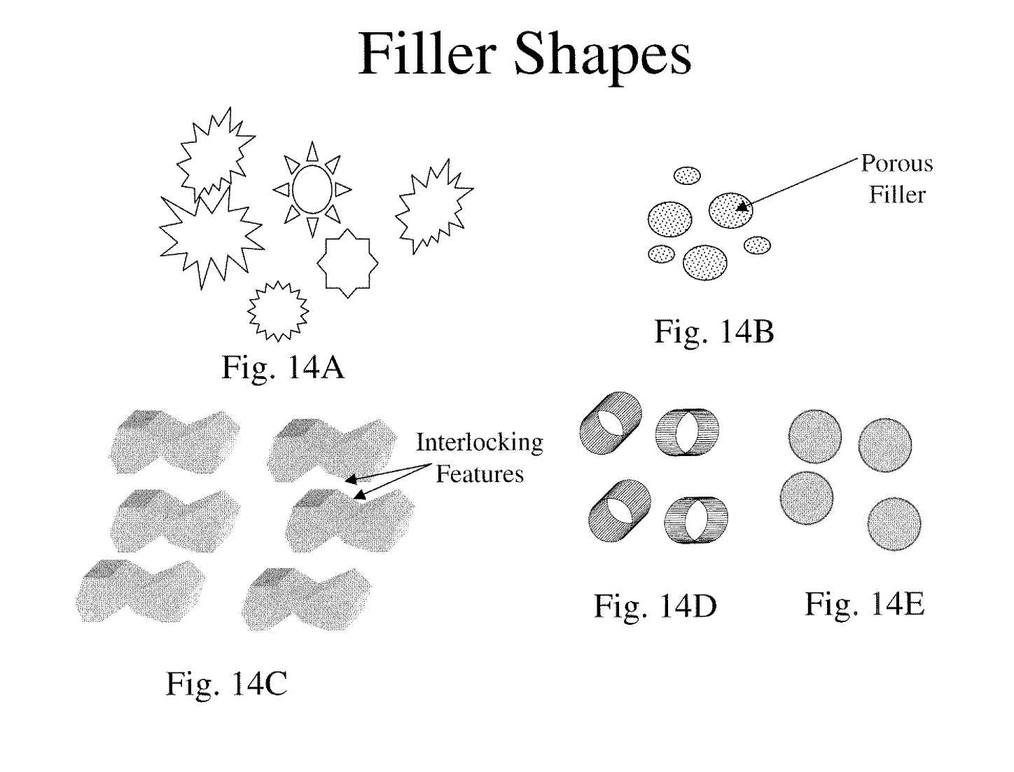

FIGS. 12A and 12B and FIGS. 13A and 13B illustrate how filler materials 24 acting as reinforcement members provide the necessary constraint to lessen disc height loss and elastomeric material deformation. In FIGS. 12A and 12B, filler materials 24 are interlocking flake shaped, more clearly depicted in FIG. 14C. In FIGS. 13A and 13B, filler materials 24 are small porous micropheres (FIG. 14B) or microspheres (FIG. 14E).

Filler materials 24 are not limited to any particular shapes and for illustrative purposes, in addition to the shapes described above, include but are not limited to, spherlites and platelets (FIG. 14A) and tubes (FIG. 14D).

The devices of this invention may be produced by a number of methods. In the event the device comprises reinforcement members that extend to the periphery of the elastomeric material as shown, for example in FIGS. 2A, 2B, 7A and 7B, the methods of laminate molding or pre-impregnation lamination may be utilized.

Laminate molding relates to a process that utilizes a two-part mold with a cavity having a shape that corresponds to the external geometry of the device. In forming an intervertebral replacement disc, an end plate is placed in both halves of the mold. The endplate or reinforcing layers may contain surfaced modifications to enhanced polymer attachment including surface finish, primers, coatings, ion bombardment, plasma treatments, for example. Preformed elastomeric materials and reinforcing layers are cut to fit within the cavity. Alternating levels of the preformed elastomeric material and reinforcement materials are placed within the cavity. Once each half of the mold is filled with the required levels of preformed elastomeric material and reinforcement material, the mold is then closed and compression applied to consolidate the preformed elastomeric material and reinforcing layers. If a thermoplastic or thermoset polymer is utilized for the elastomeric material, the mold is heated and cooled to ensure laminate incorporation and/or material cure. Conversely, a thermoset cast polymer can also be utilized for the elastomeric material by pouring/dispensing into the cavity and then applying alternating layers of the reinforcement material. Alternate means of curing the thermoset polymers known to the industry can be employed, including radiation, UV, steam, and ultrasonics, for example.

The pre-impregnation lamination technique refers to a method wherein the reinforcement members are pre-attached and/or impregnated with the elastomer before placement into the mold. This can be accomplished by several means including co-extrusion or vacuum forming. The co-extrusion method relates to formation of co-extruded sheets of reinforcing layer and elastomeric material that are extruded into a multilayer sheet which is then cut and placed into the mold cavity for attachment to the endplates. Vacuum forming is used to attach the reinforcement layers to the polymer by layering several sheets and applying a vacuum to pull the sheets and elastomer together in a cavity. The laminate sheet can then be cut and placed into the cavity for compression molding. The sheets are subsequently cut to fit and layer into the mold as described above, or the sheets can be processed to have multiple layers of elastomeric material and reinforcing members and then cut to fit for placement into the mold. Once the co-extruded sheets are in the mold, the mold is closed and then cured by techniques described above or by other known in the art curing techniques.

In the case when domed reinforcing laminate layers are used, the device of this invention can be produced by the same methods described above with a few refinements. The domed reinforcement layers are radiused or domed prior to placement into the pre-impregnation lamination mold or laminate mold. The final over molding is performed to encapsulate the subassembly.

There are several ways to make the devices of the invention when using fillers as the elastomeric core's reinforcing material. One method may be referred to as the orientated reinforcement filler method in which the relatively small particulate type fillers are oriented by methods typical in the polymer processing industry. These include controlled injection/flow by injection location at one edge of the part and gas vent at the opposite edge of the part. Flow of the polymer/filler encourages orientation of the filler in parallel to the direction of flow.

Another manufacturing technique for incorporating fillers in to the elastomeric core of the device of this invention may be described as a compression method technique. In this technique, thin sheets of uncured elastomer are covered with generally flat fillers by manual or spray application of the filler. The sheet is then gently exposed to compression flattening of the filler material unto the polymer surface. Several layers of the filler coated sheet are then combined together.

Yet another manufacturing method for incorporating fillers in to the elastomeric core of the device of this invention relates to use of cast polymers. In using cast polymers, the generally flat fillers are added to the cast polymer solution with solvents. The solvent evaporates from the mixture allowing the fillers to lay flat as the cast polymer solution forms sheets. Sheets of the cast polymer containing the fillers are then cut and placed into the cavity for final cure such as by compression molding.

It should be understood that the foregoing disclosure and description of the present invention are illustrative and explanatory thereof and various changes in the size, shape and materials as well as in the description of the preferred embodiment may be made without departing from the spirit of the invention.

What is claimed is:

1) An intervertebral disc replacement device comprising an elastomeric core comprising a top end and a bottom end and at least one reinforcement member set in a substantially horizontal orientation.

2) The device of claim 1 wherein the device further comprises a top endplate and a bottom endplate attached to the respective top and bottom ends of the elastomeric core.

3) The device of claim 1 wherein the elastomeric core comprises elastomers selected from the group of thermoset and thermoplastic elastomers.

4) The device of claim 3 wherein the elastomer is a thermoset elastomer.

5) The device of claim 4 wherein the thermoset elastomer is selected form the group consisting of polyhexene, silicone, and polyol/isocyanate urethane.

6) The device of claim 5 wherein the elastomer is polyhexene.

7) The device of claim 5 wherein the elastomer is silicone.

8) The device of claim 5 wherein the elastomer is polyol/isocyanate urethane.

9) The device of claim 3 wherein the elastomer is a thermoplastic elastomer.

10) The device of claim 9 wherein the thermoplastic elastomers is selected for the group consisting of aromatic urethanes, aliphatic urethanes, and polycarbonate block co-polymers.

11) The device of claim 10 wherein the polycarbonate block co-polymer is polycarbonate urethane.

12) The device of claim 10 wherein the polycarbonate block co-polymers is polycarbonate-silicone urethane.

13) The device of claim 1 wherein the reinforcement member comprises an elastomeric polymer.

14) The device of claim 1 wherein the reinforcement member comprises a laminate layer.

15) The device of claim 14 wherein the laminate layer is in a shape selected form the group consisting of strips, discs, squares, ovals, crescents, and sheets.

16) The device of claim 14 wherein the reinforcement members comprise a woven sheet produced from PEEK and the elastomer comprises a polycarbonate urethane.

17) The device of claim 14 wherein the reinforcement members comprise a donut-shaped sheet of titanium and the elastomer comprises a polycarbonate silicone urethane.

18) An intervertebral disc replacement device comprising an elastomeric core comprising a top end and a bottom end and filler materials.

19) The device of claim 18 further comprising a top endplate and a bottom endplate attached to the respective top and bottom ends of the elastomeric core.

20) The device of claim 18 wherein the filler materials are in shapes selected from the group consisting of flakes, platelets, spheroids, spherlites, microspheres, micro-tubes and small discs.

21) The device of claim 18 wherein the filler materials are porous.

22) The device of claim 18 wherein the elastomer is a polycarbonate urethane and the filler is porous microspheres of calcium.

23) The device of claim 18 wherein the elastomer is polycarbonate silicone urethane and the filler is an interlocking shape produced from clay.
